(19) United States
(12) Patent Application Publication
(10) Pub. No.: US 2023/0020320 A1
(43) Pub. Date: Jan. 19, 2023

(54) Liquid Composition for Cleaning, Sanitizing and/or Disinfecting

(71) Applicant: CORTICALIS AS, Oslo (NO)

(72) Inventors: Ståle Petter Lyngstadaas, Nesoddtangen (NO); Håvard J. Haugen, Oslo (NO)

(73) Assignee: CORTICALIS AS, Oslo (NO)

(21) Appl. No.: 17/939,570

(22) Filed: Sep. 7, 2022

Related U.S. Application Data
(63) Continuation of application No. PCT/EP2021/057024, filed on Mar. 18, 2021.

(30) Foreign Application Priority Data
Mar. 18, 2020 (EP) 2016-4065.3
Mar. 18, 2020 (EP) 20164065.3

(51) Int. Cl. A61K 8/22; A61K 8/90; A61K 8/64; A61Q 90/00

(52) U.S. Cl. CPC A61K 8/22; A61K 8/90; A61K 8/64; A61Q 90/00; A61K 2800/70

(57) ABSTRACT

Aspects of the present invention relate to an improved antimicrobial and/or anti-viral composition comprising an antimicrobial and/or anti viral mixture consisting of: H₂O₂ at a final concentration of between 3-7% v/v, and a poloxamer at a concentration of 0.1-10% w/v. The poloxamer is in a composite hydrogel formulation. The composition disclosed herein is antimicrobial and/or anti-viral and is particularly useful for cleaning, sterilizing, disinfecting, sanitizing, decontaminating and/or debriding biological surfaces and surfaces of biomaterials, such as when provided in a hand sanitizer, mouth wash, nasal rinse and/or for wound care and/or chronic ulcer care and use for dental prophylaxis and peri-implant health maintenance.

### FIELD

Aspects of the present invention relate to multifunctional cleaning and/or antifouling compositions comprising H₂O₂ and a composite hydrogel formulation of pluronic acid, wherein the composition is in liquid form at room temperature. The compositions described herein inhibit microbes and inflammation in a subject and are particularly useful for dermal sanitizing, oral-laryngeal sanitizing, nasal sanitizing, in oral prophylaxis and periimplantitis treatment, implant health maintenance, in periodontitis and periodontal health, in wound care or chronic ulcer care.

### BACKGROUND

Biological surfaces like skin and mucosa and/or biomaterial surfaces are in constant contact with and frequently colonized by a plethora of microorganisms and virus. They therefore frequently require cleaning and/or sanitizing with compositions that are antibacterial and/or antiviral without causing harm to the biological tissue in question and preferably without causing microbial and/or viral resistance.

In particular, the skin of the body, such as but not limited to on the hands, the feet, or the face is often exposed to microorganisms and needs to be cleaned, sterilized and/or sanitized regularly to inhibit settlement and/or transfer of pathogens and/or microbes and/or viruses.

Hand Sanitizers

A hand sanitizer is a liquid or gel, typically used to decrease infectious agents on the hands. Formulations of the alcohol-based type are preferable to hand washing with soap and water in most situations in the healthcare setting. It is generally more effective at killing microorganisms and often better tolerated than soap and water. They are typically available as liquids, gels, wipes and/or foams.

Alcohol-based versions typically contain some combination of isopropyl alcohol, ethanol (ethyl alcohol), or n-propanol. Versions that contain 60 to 95% alcohol are considered most effective. Hand sanitizers containing at least 60% alcohol or containing a “persistent antiseptic” kill many different kinds of bacteria, including antibiotic resistant bacteria and TB bacteria, but are less effective against virus.

In general, alcohol-based hand sanitizers inhibit a variety of microorganisms but not spores. Some versions contain compounds such as glycerol to prevent drying of the skin. Non-alcohol-based versions may contain benzalkonium chloride, chlorhexidine gluconate or triclosan. Care should be taken as they are flammable.

Alcohol-based hand sanitizers have been commonly used in Europe since at least the 1980s. The alcohol-based version is on the World Health Organization's List of Essential Medicines, the safest and most effective medicines needed in a health system. The wholesale cost in the developing world is about US$1.40-3.70 per bottle.

One well documented draw-back with commercially available hand-sanitizers is that the alcohol in hand sanitizers may not have the 10-15 seconds exposure time required to denature proteins and lyse cells. Further, it is often applied in too low quantities (0.3 ml) or in too low concentrations. In environments with high lipids or protein waste (such as food processing), the use of alcohol hand rubs alone may not be sufficient to ensure proper hand hygiene.

To solve this, there are new alcohol gel sanitizers marketed as alcohol rub sanitizers, which kill most bacteria, and fungi, and stop some viruses. Alcohol rub sanitizers containing at least 70% alcohol (mainly ethyl alcohol) kill 99.9% of the bacteria on hands 30 seconds after application and 99.99% to 99.999% in one minute. Still, they are notably less effective against virus.

90% alcohol rubs are more effective against viruses than most other forms of hand washing. Isopropyl alcohol will also kill 99.99% or more of all non-spore forming bacteria in less than 30 seconds, both in the laboratory and on human skin. 90% alcohol rubs are highly flammable and have a potential for abuse, but are necessary to use to kill especially viruses, including enveloped viruses such as the flu virus, the common cold virus, coronaviruses, and HIV, though is notably ineffective against the rabies virus.

Hydrogen Peroxide

Hydrogen peroxide (H₂O₂) is a very pale blue liquid which appears colourless in a dilute solution, slightly more viscous than water. It is a weak acid. It has strong oxidizing properties and is therefore a powerful bleaching agent that is mostly used for bleaching paper but has also found use as a disinfectant and as an oxidizer. Hydrogen peroxide in the form of carbamide peroxide is widely used for tooth whitening (bleaching), both in professionally- and in self-administered products.

Hydrogen peroxide is unstable and slowly decomposes in the presence of light. Because of its instability, hydrogen peroxide is typically stored with a stabilizer, typically organophosphates, sodium pyrophosphate, sodium phytate or sodium citrate, in a weakly acidic solution in a dark coloured bottle.

Hydrogen peroxide may be used for the sterilization of various surfaces, including surgical tools and may be deployed as a vapour (VHP) for room sterilization. H₂O₂ demonstrates broad-spectrum efficacy against virus and microbes including, but not limited to, bacteria, yeasts, and bacterial spores. In general, greater activity is seen against Gram-positive than Gram-negative bacteria though; however, the presence of catalase or other peroxidases in these organisms may increase tolerance in the presence of lower concentrations. Higher concentrations of H₂O₂ (10 to 30% v/v) and longer contact times are sometimes required for sporicidal activity.

Hydrogen peroxide is seen as an environmentally safe alternative to chlorine-based bleaches, as it degrades to form oxygen and water and is generally recognized as safe as an antimicrobial agent by the U.S. Food and Drug Administration (FDA).

Historically, hydrogen peroxide was used for disinfecting wounds. Today, it is thought to inhibit healing and to induce scarring, because it destroys newly formed skin cells at high concentrations. This is understood to be caused by the drying-out effect of peroxide. One study found that only very low concentrations (0.03% v/v solution, this is a dilution of typical 3% v/v Peroxide by 100 times) may induce healing, and only if not applied repeatedly. A 0.5% v/v solution was found to impede healing. Recently, new studies have however shown that peroxide is an important and intrinsic part of the cellular signalling and defence system, generating a signal that activates local defence cells in response to infections and insults. In line with this it is also now known that cells employ peroxide directly to kill microbes and virus in their immediate vicinity. Because of this knowledge, peroxide has gained renewed attention as a biologically active sanitizing substance.

Pluronic Acid

Pluronics® or poloxamers are tri-block copolymers of poly(ethylene oxide)-poly(propylene oxide)-poly(ethylene oxide) (PEO-PPO-PEO). This group of synthetic polymers is thermo-reversible in aqueous solutions. The sol-gel transition is governed by the composition, molecular weight and concentration of each constituent block polymer. The hydrophilic ethylene oxide and the hydrophobic propylene oxide give pluronics an amphiphilic structure—meaning it has a polar, water-soluble group attached to a nonpolar water-insoluble hydrocarbon chain. Amphiphilic block copolymer molecules self-assemble into micelles (a packed chain of molecules) in aqueous solution. Micelle formation is temperature dependent and affects the degradation properties of the biomaterial: below a certain characteristic temperature, known as the critical micelle temperature, both the ethylene and propylene oxide blocks are hydrated, and the PPO block becomes soluble.

Pluronics can be found either as liquids, pastes or solids. Due to their amphiphilic characteristics (presence of hydrophobic and hydrophilic components), pluronics possess surfactant properties which allow them to interact with hydrophobic surfaces and biological membranes. Being amphiphilic also results in the ability of the individual block copolymers, known as unimers, to combine and form micelles in aqueous solutions. When the concentration of the block copolymers is below that of the critical micelle concentration (CMC), the unimers remain as molecular solutions in water. However, as the block copolymer concentration is increased above the CMC, the unimers will self-assemble and form micelles, which can take on spherical, rod-shaped or lamellar geometries. Their shapes depend on the length and concentration of the block copolymers (i.e. EO and PO), and the temperature. Micelles usually have a hydrophobic core, in this case the PO chains, and a hydrophilic shell, the EO chains.

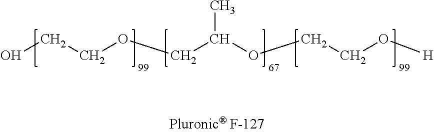

Pluronic F-127, also known as Poloxamer 407, is often used in tissue engineering because of the commercial availability of a consistent product that will undergo a sol-gel transition near physiological temperature and pH. A disadvantage of Pluronic F-127 is its fast degradation rate in vivo. To overcome this problem, Pluronic F-127 is frequently crosslinked with another α-hydroxy or amino acid in order to alter the chemical structure of its depsipeptide unit.

Pluronic acids form thermo-sensitive hydrogels, which are typically stabilized by addition of high-molecular-weight acids, such as hyaluronic acid.

Studies have documented the positive effects of pluronic acid formulations in reducing inflammation, protect tissues against damage and hinder microbial adhesion. Furthermore, it is EMA and FDA approved, completely biocompatible and safe to use clinically with no known harmful effects in human cells.

Still, it is hard to apply as a cleaning agent because it will automatically form a stable gel at over 18° C., or even between temperatures of 12-20° C. (see FIG. 1) dependent of its concentration, which makes it unsuitable to use in small passages and/or for use on rough surfaces or at body temperature and does not lend it to application with a syringe.

Accordingly, the need for additional cleaning and/or sterilizing and/or debriding compositions, which can be used to disinfect, sterilize or clean, prevent fouling and/or refouling of a fouled biological surface and/or a biomaterial surface, such as the mucosa of the oral or nasal cavity of a subject, or the exposed outer skin of a subject, such as hands and feet, is manifest.

### SUMMARY

Aspects of the present invention relate to an antimicrobial and/or anti-viral composition for cleaning and/or sterilizing a biological surface and/or a biomaterial surface e.g. in situ, comprising at least two components:
(a) H₂O₂ at a final concentration of between 0.1-7% v/v, and
(b) a composite hydrogel formulation of pluronic acid at a concentration of 0.1-10% w/v.

The composite hydrogel formulation is typically a poloxamer. Thus, a composition of the present invention comprises an antimicrobial and/or anti viral mixture consisting of:
(a) H₂O₂ at a final concentration of between 3.0-7.0% v/v, and
(b) a poloxamer at a final concentration of 1.0%-10.0% w/v.

Typically, an antimicrobial and/or anti-viral composition as disclosed herein comprises a composite hydrogel formulation of component (b), which is a poloxamer, such as pluronic acid at a concentration of 0.1-5% w/v, such as at a concentration of at the most 5% w/v, such as at a concentration of between 1.0%-5.0% w/v, such as 0.1, 0.5, 1.0 or 1.5% w/v. The composition according to the present invention in one aspect comprises a poloxamer at a concentration of 5.0% w/v. The poloxamer can be a mixture of poloxamers. Component (b) can be selected from the group consisting of pluronic acid, Pluronic® F-127 and Poloxamer 407. In a presently preferred aspect, the poloxamer is Poloxamer 407.

An antimicrobial and/or anti-viral composition in some alternatives further comprises a H₂O₂ of component (a), which has a final concentration of 0.1-7% v/v, such as a final concentration of 0.5-3.0% v/v. In one aspect, the H₂O₂ of component (a) has a final concentration of 3% v/v.

In one aspect, component (a) and component (b) are provided in a 1:1 ratio.

An antimicrobial and/or anti-viral composition in some alternatives can further comprise water and/or physiological saline, oils, preservatives flavors and/or fragrances.

In one aspect, an antimicrobial and/or anti-viral composition comprises the at least two components:

(a) H₂O₂, and (b) a composite hydrogel formulation, comprising a poloxamer, such as pluronic acid, which are kept separate from each other until they are simultaneously mixed and applied to a biological surface and/or a biomaterial surface in situ.

According to that aspect, an antimicrobial and/or anti-viral composition comprises a separate component (a), which is a composition that comprises H₂O₂ at a concentration of at least 10-50% v/v.

A composition according to some alternatives can further comprise an additional antimicrobial substance. A composition according to some alternatives can further comprise a bioactive substance, typically selected from the group consisting of peptides, drugs, bioactive ions, small molecules, radioactive molecules, and radio-opaque molecules or any combination thereof.

A composition according to some alternatives typically has a shelf-life of at least 1 years at room temperature (RT).

A composition according to some alternatives is useful for sterilizing or sanitizing or cleaning a biological surface and/or a biomaterial surface in situ.

Typically, one or more of the compositions described herein can be used to clean, sterilize, or otherwise rid the skin and/or mucosa of a subject, preferably a human, from microbes, including but not limited to bacteria or virus, such as Riboviria, Coronaviridae, or Orthocoronavirinae.

In a presently preferred aspect, one or more of the compositions described herein are used to clean or sterilize skin and/or mucosa from a Coronavirus, such as SARS-CoV-2.

In consequence, aspects of the present invention relate to the application of one or more of the antimicrobial and/or anti-viral compositions described herein as a hand wash, oral wash and/or for nasal and/or sinus cleansing, so as to remove, sterilize, or inhibit a Coronavirus, such as SARS-CoV-2 from the skin or mucosa of a subject, preferably a human.

Aspects of the present invention also concern the use of one or more of the compositions described herein for cleaning and/or sanitizing a biological surface and/or a biomaterial surface, such as in particular an implant in situ and/or a surface in the oral cavity and/or any surface covered by skin or mucosa.

In some alternatives, one or more of the compositions described herein are incorporated in a kit, wherein the at least two components, H₂O₂ and pluronic acid, are optionally kept separate such that a user may mix the two components just prior to application.

The invention consequently also relates to a method of sanitizing, sterilizing, disinfecting and/or decontaminating a biological surface and/or a biomaterial surface comprising applying the composition according to the present invention to a biological surface and/or a biomaterial surface, as well as a method of cleaning skin and/or mucosa of a subject from microbes comprising applying the composition according to the present invention to the skin and/or mucosa of a subject. The microbes comprise bacteria or virus or both, such as, but not limited to Riboviria, Coronaviridae, Orthomyxoviridae, Caliciviridae or Reoviridae or any combination thereof. The microbes comprise Orthocoronavirinae, such as, but not limited to Coronavirus, Rotavirus, Norovirus or Influenza virus type A, B, C or D or any combination thereof.

### DEFINITIONS and ABBREVIATIONS

Aspects of the present invention provides compositions and methods to clean and/or sanitize a biological surface and/or a biomaterial, such as an implant surface.

In the present context, a biological surface refers to any surface covering an anatomical structure and/or tissue, such as skin including palmar and plantar skin, oral and nasal mucosa, gastrointestinal mucosa, laryngeal, tracheal and bronchial mucosa, vaginal mucosa, penile mucosa, hair, nails, fasciae, membranes, synovial membranes, dental enamel or dental cementum. The term also encompasses a wound surface, such as in, on and/or surrounding, but not limited to, an ulcer, acute wound, wound from trauma, surgical wound, puncture wound, abrasive wound, infected wound including herpes ulcer, papilloma, chancre and acne, erosive wound, burn, blister, scab, leg ulcer, diabetic ulcer and/or chronic ulcer.

In the present context, the term “biomaterial or implant surface” typically refers to a surface of a medical and/or dental implant.

In the present context, the term “medical implant” includes within its scope any device intended to be implanted into and/or attached to the body of a vertebrate animal, in particular a mammal such as a human, for preservation and restoration of the function of the body, particularly a prosthesis of any kind, metallic and polymer-based implants in the vascular system or in the musculoskeletal system or in joints and bones, including for alleviation of pain in these structures. Non-limiting examples of medical implants are leg, arm and hand prosthesis, facial prosthesis, eye prosthesis, ileostomy devices, intrauterine devices, pacemakers, electrodes, artificial vascular structures, stents, cochlear implants, hip-joint prostheses, knee prostheses, elbow prostheses, finger prostheses, cochlear prostheses, or fixation screws.

In the present context, the term “dental implant” includes within its scope any device intended to be implanted into the oral cavity of a vertebrate animal, in particular a mammal such as a human, for example in tooth and jaw restoration procedures. Dental implants are herein selected from the group consisting of: Implants, bars, bridges, abutments, crowns, caps, dental fillings and prosthetic parts in the oral cavity. Dental implants may also be denoted as dental prosthetic devices. Generally, a dental implant is composed of one or several implant parts. For instance, a dental implant usually comprises a dental fixture coupled to secondary implant parts, such as an abutment and/or a dental restoration such as a crown, bridge or denture. However, any device, such as a dental fixture, intended for implantation may alone be referred to as an implant even if other parts are to be connected thereto.

In a second embodiment, the term biomaterial surface includes the surface of any medical device or tool, disposable or not, designed to come in temporary contact with living tissue, such as, but not limited to, surgical instruments, electrodes, scalpels, probes and gauges, catheters, syringes, scissors, needle holders, contact lenses, wound dressings, band aids, transdermal fixing devices, and/or diagnostic tools like ultrasound devices, x-ray machines and/or imaging equipment, e.g. various scopes, surgical cameras, impression materials or intraoral scanners.

In the present context, the term peroxide is used interchangeably with Hydrogen peroxide and/or (H₂O₂).

A microorganism, or microbe, is a microscopic organism, which may exist in its single-celled form or in a colony of cells. Microorganisms include all unicellular organisms and so are extremely diverse. All of the Archaea and Bacteria are microorganisms (Prokaryotes). Some protists are related to animals and some to green plants. Many of the multicellular organisms are microscopic, namely micro-animals, some fungi and some algae.

An antimicrobial is an agent that kills microorganisms or stops or inhibits their growth. Antimicrobial medicines can be grouped according to the microorganisms they act primarily against. For example, antibiotics are used to treat or inhibit bacteria, and antifungals are used to treat or inhibit fungi. They can also be classified according to their function. Agents that kill microbes are microbicidal, while those that merely inhibit their growth are called biostatic both are included in the term“antimicrobial”. The use of antimicrobial medicines to treat or inhibit infection is known as antimicrobial chemotherapy, while the use of antimicrobial medicines to prevent infection is known as antimicrobial prophylaxis.

A virus is a small infectious agent that replicates only inside the living cells of an organism. Viruses can infect all types of life forms, from animals and plants to microorganisms, including bacteria and archaea.

Antiviral drugs are a class of medication used specifically for treating or inhibiting viral infections rather than bacterial ones. Most antivirals are used for specific viral infections, while a broad-spectrum antiviral is effective against a wide range of viruses. Unlike most antibiotics, most antiviral drugs do not destroy their target pathogen; instead they inhibit their replication and/or development.

Antiviral drugs are one class of antimicrobials, a larger group which also includes antibiotic (also termed antibacterial), antifungal and antiparasitic drugs, or antiviral drugs based on monoclonal antibodies. Most antivirals are considered relatively harmless to the host, and therefore can be used to treat or inhibit infections. They should be distinguished from viricides, which are not medication but deactivate or destroy virus particles, either inside or outside the body. Natural antivirals are produced by some plants such as eucalyptus or Australian tea trees.

As used in the present context, the term “antimicrobial”, refers to a composition that is effective against microbes and virus. In its broadest meaning, one or more of the compositions described herein are antimicrobial, e.g., antibacterial or antiviral, a bactericide or a viricide.

As used herein, the term “about” or “approximately” means within an acceptable error range for the particular value as determined by one of ordinary skill in the art, which will depend in part on how the value is measured or determined e.g., the limitations of the measurement system.

### BRIEF DESCRIPTION OF THE DRAWINGS

FIG. 1: Gel boundaries for aqueous saline (physiological conditions) solutions of copolymer F-127. The filled circles are data points obtained by the tube inversion method for the mixture. The unfilled squares are data points from rheometric analyses. The chart clearly indicates that pluronic concentrations below 15% w/v in saline remains fluid independent of temperature. Fluid pluronic solutions form independent micelles, but the concentration is not high enough for the pluronic micelles to assemble into a cubic gel. Thus, low pluronic concentrations that allow for micelle formation but stay liquid and act as detergent may be good for tissue-friendly cleaning of biological surfaces, but addition of other reactants that can contribute to the decontamination is expected to completely disrupt the micelle formation.

FIG. 2: Schematic drawing depicting the synergistic effects from mixing pluronic with peroxide. During the interaction with pluronic, the peroxide generates active oxygen (ROS) degrade microbial and viral nucleic acids and membranes, and kills bacteria, including anaerobes, and viruses. The peroxide also drives pluronic micelle dissolution that is countered by the strong tendency of pluronic to form micelle structures. The dynamic equilibrium between solubilized and micelle pluronic allow for an active entrapment of dirt, organics and contaminants into the micelle structure. The liquid hydrophilic nature of the pluronic hydrogel also promotes easy access for the ROS to wrinkles and crevices, unhindered by foaming. Moreover, the pluronic hydrogel moisture the skin and the oxygen released from the peroxide helps the wound to heal. The ROS also has the ability to clean and reactivate (make the surface hydrophilic) titanium, one of the most widely used metal in medical and dental implants. The effect of ROS alone is too brief to have a significant impact on these processes and would not remove the contaminants or provide moisture. Pluronic alone for stable micelles that is far less effective in contaminant entrapment and do not kill microbes or degrade viral nucleic acids. Mixed together in the right concentration pluronic micelle formation is more dynamic, moistening and oxygenation is optimized, and the peroxide action is prolonged and increased and kept at the surface where it is needed.

FIG. 3: Titanium surfaces contaminated with S. epidermidis bacteria was cleaned with growth medium (BHI, Negative control), H₂O₂ 5% v/v+f-127 1% w/v (Test 2), H₂O₂ 5% v/v+f-127 7% w/v (Test 4), Chlorhexidine 0.2% w/v+f-127 7% w/v (Test 5) or Ethanol 75% v/v+f-127 1% w/v. The negative control did not remove the bacteria, nor was the bacteria destroyed. The peroxide+pluronic acid mix killed all bacteria, dissolved the biofilm and removed the debris almost completely (Tests 2 & 4). Test 4 was slightly, but not statistically significant, more effective than Test 2. Chlorhexidine (Test 5) and Ethanol (Test 6) did kill most of the bacteria but did not remove any debris from the surface, leaving the surfaces heavily contaminated. Surfaces visualized after cleaning in a scanning electron microscope. Scale bar is 20 micrometres.

FIG. 4: Titanium surfaces contaminated with S. epidermidis bacteria was cleaned with A) saline (Negative control), B) Chlorhexidine 0.2% w/v, or C) H₂O₂ 3% v/v+f-127 pluronic Acid 5% w/v. After cleaning the surfaces were stained with fluorescent dyes, green for living cells and red for dead cells. The negative control (saline) did not remove any of the bacteria, nor was the bacteria destroyed. The chlorhexidine killed all bacteria but left them to contaminate the surface. The hydrogen peroxide+pluronic acid mix killed all bacteria, dissolved the biofilm and removed the debris almost completely Surfaces visualized in a confocal microscope after cleaning and staining.

FIG. 5: Regrowth of S. epidermidis bacteria, harbouring the luciferase gene making them fluorescent, on sanitized titanium surfaces. The results show that D) water with detergent (SDS, 0.1%), E) water with 5% pluronic acid and F) saline had minimal sanitizing effect, as regrowth of the biofilm occurred within 3-4 hours after cleaning. Chlorhexidine, 0.2%, on the other hand killed all bacteria with no regrowth observed although the dead biofilm stuck and remained on the surface (see FIGS. 3 and 4). Hydrogen peroxide, 5% v/v, alone (A) cleaned the surface well as seen by a delay of about 13 hours before the biofilm restored itself. However, hydrogen peroxide 3% v/v combined with 5% pluronic acid in water cleaned the surface significantly better, delaying regrowth with more than 15 hours.

FIG. 6: Left and right thumbs were sanitized four times daily for five consecutive days with a mix of A) pluronic acid (5% w/v) and hydrogen peroxide (3% v/v) or B) Antibac Pharma™ containing ethanol and propan-2-ol respectively. After the sanitizing period both fingers were stained with iron oxide to visualize fissures and cracks. Fissures and cracks are easily visible as brown striated stains on the Antibac Pharma™ treated thumb, whereas the pluronic-peroxide formulation caused no harm to the skin of the left finger.

FIG. 7: Fingers were marked with a line of permanent ink from a laboratory marker that was subsequently washed off with various sanitizing/decontaminating solutions to visualize their ability to remove contaminants from the skin. Finger A) was washed only with water. Finger B) was cleaned with warm water and soap. Finger C) was cleaned with 0.2% chlorhexidine and finger D) was rinsed with Pyrispet™ (0.1% cetyl-pyridinium chloride). Finger E) was cleaned with Antibac Pharma™ (Alcohol based sanitizer), finger F) was cleaned with bleach (4% Sodium hypochlorite). Fingers G) and H) were rinsed with hydrogen peroxide (1% and 5% v/v in water respectively). Finger 1) was washed in a solution of pluronic acid in water (5% w/v). Finger J) was sanitized with a mix of pluronic acid (5% w/v) and hydrogen peroxide (1% v/v). The results show that bleach and hydrogen peroxide is effective in removing the ink stain, but that the most efficient cleaning is obtained by the combination of pluronic acid and hydrogen peroxide.

### DETAILED DESCRIPTION

There has been a long felt need in community for the presently described compositions, which effectively clean, disinfect and/or sanitize a biological surface and/or a biomaterial surface. e.g., including but not limited to skin or mucosa, such as a surface in the oro-laryngeal or nasal cavity. Any one or more of the compositions described herein are useful for the rapid and effective treatment or inhibition of bacteria or virus or both essentially without leaving contaminating material residues.

The presently described compositions are water soluble and easy to rinse off, tissue-friendly non-ionic surfactant. These compositions comprise a non-toxic formulation of well-studied active ingredients in clinical use, which is particularly suitable for injectable, oral and/or cutaneous applications. The herein for the first time described composition is proven to be non-sensitizing and non-irritating in clinical tests. The compositions described herein are compatible with most other therapeutic agents useful against biofouling and inflammation.

The compositions described herein have a liquid state at room temperature that allows trouble-free mixing and application. The compositions have an easy flowing liquid consistency with a surfactant effect that allows the compositions to reach difficult places when applied into narrow crevices, defects and/or folds on skin and/or mucosa.

Without being bound to a particular theory or mechanism of action, it is contemplated that he compositions described herein mimic the natural release of reactive oxygen species (ROS) from peroxide produced by human cells. The charge from the reactive oxygen destroys microbial membranes and the oxygen itself is also toxic to anaerobic bacteria. The human cells themselves are protected against ROS by enzymes in their cell membrane and the local tissue can benefit from the increase in oxygen. Microbes have no such protection, nor can they develop resistance because of fundamental differences in their cell-membrane design. Thus, the composition can effectively dissolve biofilm, debris and mineral deposits, as well as, dissolve extracellular organics at the application site.

Through the active oxygen released from the liquid hydrogel, the use of the herein presented compositions also remove carbon contamination from a titanium containing implant surface and reactivate the titanium dioxide layer of the implant. This process re-establishes the original charge and hydrophilicity of the implant, restoring its optimal biological surface properties. This is a factor for further survival, or even successful reintegration, of said treated implant. The present compositions provide an advanced micelle forming gel formulation that works in synergy with natural occurring oxygen to break down and remove biofouling, eliminate microbes, keep tissue and implant moist and reactivate titanium implant surfaces.

The hydrogel component of the compositions and the active oxygen work in synergy to avoid foaming from oxygen release and to keep activated oxygen in place at the surface for a prolonged biological and chemical effect. The use of any one or more of the compositions set forth herein provides moisture and allows the charged oxygen to work without risk of drying out skin, tissue and/or implant surfaces, during which the active oxygen eradicates microbes that are then suspended and entrapped inside the micelle-forming hydrogel. Organic contaminants are in turn denatured by the strong detergent effect, broken down by the active oxygen and dissolved and entrapped in the hydrogel. Both hydrogel and oxygen reduce inflammation and support tissue health. The active oxygen released in turn strengthens the cellular defence network. In synergy, the gel and the active oxygen both removes contaminants, acts as an antimicrobial and viricidal agent and reactivates a titanium implant surface. The interaction between the pluronic micelle formation and peroxide potentiates micelle formation and increases the cleaning effect significantly.

The presently described compositions provide a novel formula of a biocompatible hydrogel-peroxide combination with strong, antimicrobial, viricidal and non-ionic detergent properties with improved micelle dynamics for solubilization and entrapment of debris and microbes for effectively cleaning, sanitizing, sterilizing and/or debriding a biological surface and/or a biomaterial surface in situ. It is easily rinsed off with a towel, wipe or by water, but it can also be left to air-dry and will completely decompose to water, oxygen and carbon dioxide.

The presently disclosed compositions provide a novel and improved approach to cleaning, sanitizing, sterilizing and/or disinfecting skin, mucosa and/or implant surfaces. In particular alternatives, for example, any one or more of the compositions described herein are incorporated into a mouth wash and/or nasal rinse and these formulations can be used to prevent, treat, inhibit, or ameliorate, an oral and/or nasal disease (e.g., dental caries, periodontitis, gingivitis, mucositis, peri-implantitis, sinusitis and/or rhinitis). In additional alternatives, said mouth rinses and/or nasal rinses can be used in methods to inhibit bacterial or viral infections, such as SARS-CoV-2 in the oral or nasal mucosa of a subject, such as a human.

The desired formulations set forth herein are based on the combination of hydrogen peroxide (H₂O₂) and pluronic acid in such a concentration that the resulting composition is in a watery liquid form at physiological temperatures. The H₂O₂ component of the composition can either be in the form of a concentrate (at a concentration of at least 10-50% v/v.) in a separate vial for mixing immediately before use, or provided as an emulsion or dissolved directly into a hydrogel, said hydrogel at least partly consisting of pluronic acid and water and/or physiological saline, with a typical final concentration of 0.5-7%. The pluronic component itself can be any one or a combination of the pluronic acids, e.g. the F-127 variety. The concentration of pluronic acid is typically 0.1-10% w/v, such as 0.1-7% w/v, such as 0.1-2.5% w/v. That is, the concentration of pluronic acid in any one or more of the compositions described herein can be at least or equal to 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, or 10% w/v or a percentage w/v that is within a range defined by any two of the aforementioned percentages.

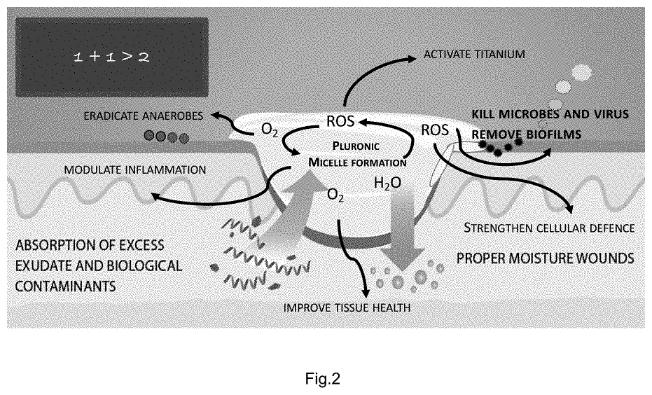

Pluronic-acid works both as solubilizer and detergent in the composition disclosed herein, as well as a moisturizer. In the presence of peroxide, the hydrogel formulation establishes a dynamic micelle forming solution. This dynamic state facilitates efficient solubilization and entrapment of particles, microbes and pollutants during the cleaning procedure (see e.g. FIG. 2).

The present innovation is based on a synergetic effect between pluronic acid and peroxide. Pluronic acid has an ability to form micelles. This ability usually increases with increasing temperature and is shifted toward a gel state at physiological conditions (such as at >20 degrees Celsius). The peroxide addition allows the pluronic acid to stay in the liquid state even at physiological temperature.

When mixed with hydrogen peroxide, the present inventors for the first time disclose that the micelles are more dynamic and less stable and in a “dynamic” equilibrium with the peroxide radical activity even under high temperature. In effect, this means that in the presence of peroxide, the micelle structure is dissolved and reforms constantly also when the gel is applied onto human tissue, skin and/or mucosa. This effect keeps the hydrogel liquid also at high temperatures and increases the detergent, denaturation and entrapment effect of the pluronic acid significantly.

Combined with the effect of hydrogen peroxide to release free oxygen radicals on viral particles, microbes and necrotic tissue, the micelle transitions dissolve and entrap organic contamination that are then removed when the gel dries off, is wiped off, or washed off.

Desirably, the addition of hydrogen peroxide to the pluronic acid in the compositions described herein increases the temperature at when gelation occurs. e.g., that the pluronic gel, containing low concentrations of peroxide, is liquid at room temperate and thus can be applied through a syringe needle or from a dispenser bottle without clogging the nozzle. This is not possible with pluronic acid alone because it starts forming a stable “packed micelle” gel already at room temperature, even at low concentrations when in narrow compartments and/or at the “vapor front” at the nozzle of the dispenser, thus is very hard to squeeze through a narrow tip of a syringe, or a pump applicator from a dispenser bottle. pluronic gel for wound care is therefore sold as gel in a box or a tube. The increased effect from the combination of peroxide and pluronic acid was unexpected and surprising.

The liquid formulation of the pluronic hydrogel in combination with peroxide also assists during application. It enables the cleaning and/or sanitizing composition to reach folds, wrinkles, crevices, fissures, narrow spaces and/or undercuts that a pluronic gel alone cannot reach because of its gel forming nature making it viscous at physiological temperatures. Thus, it is more efficient in cleaning rough (implant) surfaces, narrow spaces such as folds, fissures and wrinkles on skin and mucosa and between teeth.

Compositions

Aspects of the present invention relate to novel antimicrobial and/or anti-viral compositions, which can be used for cleaning, sanitizing, sterilizing, disinfecting and/or decontaminating a biological surface and/or a biomaterial surface in situ. In some alternatives, the aforementioned compositions comprise two components:
a. H₂O₂ at a final concentration of between 0.1-7% v/v, and
b. a composite hydrogel formulation of pluronic acid at a concentration of 0.1-10% w/v.

In some of the aforementioned compositions, the final concentration of H₂O₂ is at least or equal to 0.1%, 0.5%, 1.0%, 1.5%, 2.0%, 2.5%, 3.0%, 3.5%, 4.0%, 4.5%, 5.0%, 5.5%, 6.0%, 6.5%, or 7.0% w/v or a percentage w/v that is within a range defined by any two of the aforementioned percentages. In some of the aforementioned compositions, the final concentration of pluronic acid is at least or equal to 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, or 10% w/v or a percentage w/v that is within a range defined by any two of the aforementioned percentages.

Preferably, said compositions are formulated so that they remain a liquid at physiological temperatures, such as at a temperature of at the most 40° C., such as at a temperature between 20-40° C., such as at 37° C. e.g., 20, 22, 24, 26, 28, 30, 32, 34, 36, 38, or 40° C. or at a temperature that is within a range defined by any two of the aforementioned temperatures.

Some of the presently disclosed compositions are characterized in that they comprise a low concentration of pluronic acid, which allows the composition to be in a liquid state in high temperature instead of in a gel-state, which a desirable feature for it to be usable for cleaning, sanitizing, sterilizing, disinfecting and/or decontaminating a biological surface and/or a biomaterial surface in situ. In particular, the liquid state of the hydrogel is desired when the composition is incorporated into a hand sanitizer, hand wash, oral wash and/or for nasal and/or sinus cleansing solution, in particular for washing virus off of skin and mucosa.

The presently disclosed compositions comprise the at least two components (a) and (b) in such a ratio that the composition is in a liquid state at physiological temperatures instead of in a more viscous or gel-like state. Typical ratios of concentrations between component (a) and component (b) are approximately 1:1 (concentration of H₂O₂: concentration of pluronic acid). In general, the higher the concentration of the pluronic acid, the higher the concentration of the H₂O₂ is needed to keep the composition in a liquid state at temperatures of between 25-40° C. However, too high concentrations of peroxide will destroy the pluronic acid amphiphilic properties and disrupt micelle formation, rendering it useless as a detergent and/or a desaturating agent.

In one alternative of an antimicrobial and/or anti-viral composition according to the present invention, the composite hydrogel formulation of component (b) comprises pluronic acid at a concentration of 0.1-10% w/v, such as at a concentration of 10% w/v, such as of 0.1, 0.5, 1, 1.5, 2, 2.5, 3, 3.5, 4, 4.5, 5, 5.5, 6, 6.5, 7, 7.5, 8, 8.5, 9 or 9.5% w/v or an amount that is within a range defined by any two of the aforementioned concentrations.

In another alternative of an antimicrobial and/or anti-viral composition according to the present invention, the composite hydrogel formulation of component (b) comprises pluronic acid at a concentration of at the most 10% w/v, such as at the most 0.1, 0.5, 1, 1.5, 2, 2.5, 3, 3.5, 4, 4.5, 5, 5.5, 6, 6.5, 7, 7.5, 8, 8.5, 9 or 9.5% w/v or an amount that is within a range defined by any two of the aforementioned concentrations.

An antimicrobial and/or anti-viral composition according to the present invention can be a composition wherein the H₂O₂ of component (a) has a final concentration of 0.1-7% v/v, such as 0.5-3% v/v, such as 0.1-5% v/v. In one embodiment, the H₂O₂ of component (a) has a final concentration of no more than 7% v/v, such as 0.1-7% v/v, such as 1, 2, 3, 4, 5, 6 or 7% v/v.

An antimicrobial and/or anti-viral composition according to the present invention can further comprise water and/or physiological saline.

The two components of the antimicrobial and/or anti-viral composition according to the present invention can be in one solution or the at least two components can be kept separate from each other such that they can be simultaneously mixed and applied to a biological surface and/or a biomaterial surface in situ.

In an antimicrobial and/or anti-viral composition according to the present invention, wherein the components are kept separate from each other before application, the separate component (a) can be a composition that comprises H₂O₂ at a concentration of at least 10-50% v/v, such as at the most 10, 20, 30, 40 or 50% v/v. In one alternative, a composition of the present invention comprises H₂O₂ at a concentration of 30% v/v.

Emulsifier(s) and/or Viscosity Modifier(s)

In some alternatives, the antimicrobial and/or anti-viral composition according to the present invention further comprises one or more emulsifier(s) and/or viscosity modifier(s). Said emulsifier and/or viscosity modifier may be selected from the group consisting of glycerine, glycols, polyethylene glycols (PEG), polyoxyethylene polyoxypropylene block copolymer (pluronic polyols), polyglycol alginate (PGA), CMC (carboxyl methyl cellulose), glycerol, Aloe Vera gel, alginate, hyaluronic acid (HA) and chitosan or any combination thereof.

The antimicrobial and/or anti-viral composition according to the present invention may also comprise one or more detergent(s) selected from the group consisting of SDS (sodium dodecyl sulphate), sodium stannate, sodium pyrophosphate, oxine and SLS (sodium lauryl sulphate) or any combination thereof.

The antimicrobial and/or anti-viral composition according to the invention may further comprise one or more fragrance or flavouring oil(s), such as, but not limited to perfumes and oils of spearmint, peppermint, wintergreen, sassafras, clove, sage, eucalyptus, marjoram, cinnamon and methyl salicylate or menthol or any combination thereof.

The antimicrobial and/or anti-viral composition according to the invention may further comprise one or more weak acidic buffers.

The antimicrobial and/or anti-viral composition according to the invention may further comprise one or more stabilizers such as, but not limited to organophosphates, carboxylate salts, sodium pyrophosphate, sodium phytate, colloidal stannate or sodium citrate or any combination thereof.

Bioactive Substance

Alternatively, or in addition, one or more of the compositions according to the present invention can comprise a bioactive substance, typically selected from the group consisting of EMD, peptides, drugs, bio active ions, small molecules, radioactive molecules, antimicrobial molecules and radio-opaque molecules or any combination thereof.

Cleaning Components and Antimicrobial Substances

What is more, one or more of the compositions according to the present invention can also comprise an additional antimicrobial substance and/or cleaning component.

In the present context, an additional antimicrobial substance provided in one or more of the compositions according to the present invention can be selected from the non-exclusive list consisting of amoxicillin, doxycycline, cephalexin, ciprofloxacin, clindamycin, metronidazole, azithromycin, sulfamethoxazole and trimethoprim or any combination thereof.

In one aspect, a further antimicrobial substance provided in one or more of the compositions according to the present invention is an alcohol, tetracycline, doxycycline, macrolides, penicillin (stabilized), chlorhexidine, chloramines or mixtures thereof.

In one aspect, one or more of the compositions according to the present invention comprises an additional anti-viral substance.

In one aspect, one or more of the compositions according to the present invention comprises an additional anti-inflammatory, antiphlogistic, coagulating, anesthetic and/or pain-killing agent or substance.

In one aspect, one or more of the compositions according to the present invention comprises one or more of prophylactic and/or anti-tooth-decay agents, such as, but not limited to, hydrogen fluoride, sodium fluoride, or stannous fluoride or any combination thereof.

Shelf-Life of at Least 1 Years in Room Temperature (RT)

A composition according to the present invention in one aspect has a shelf-life of at least 1 years at room temperature (RT).

A Kit

Aspects of the present invention also relate to a kit comprising a composition described herein, wherein said kit comprises at least two containers comprising said separated components (a) and (b), respectively, a syringe and a vial, a connector device, an applicator tip and an instruction leaflet and, optionally a mixing device and a cleaning tool, such as but not limited to a brush. Said kit can provide the two components (a) and (b) in a two-chamber syringe, in which case the kit further can comprise an instruction leaflet, a mixing device, an applicator tip and a cleaning tool, such as but not limited to a brush and/or a wipe.

An antimicrobial and/or anti-viral composition according to the present invention can be mixed before application and eventual storage or stored separately and mixed directly or shortly before and/or at the time of application. The application therefore, in another aspect is directed to a kit comprising a first container comprising component (a), a second container comprising component (b), and optionally at least one more (third or forth etc.) container comprising a component (c), (d), (e) etc.), which can e.g. comprise paper and/or cotton wipes, such as but not limited to wet-wipes, microparticles and/or a mesh-forming substance and/or a bioactive substance and/or a cleaning component and/or a further antimicrobial and/or anti-viral substance.

Optionally such a kit may also comprise instructions for the preparation of the composition of the invention. The kit may also comprise one or more device(s) for the application of the composition to a subject. Such a device may e.g. be a dispenser bottle, a blister pack, a double blister pack, a syringe and/or an implant cleaning and/or sanitizing tool for cleaning and/or sanitizing an implant, such as in the oral cavity.

A kit of the invention may also comprise one or more of the compositions described herein in one or more container(s) and an implant cleaning and/or sanitizing tool for cleaning and/or decontamination of an implant in the oral cavity.

In a presently preferred aspect, a kit comprising a composition according to the present invention typically provides the two components (a) and (b) in a two-chamber device that allows for easy mixing of the components prior to and/or or during application. Typically, such a device can be a two-chamber syringe, a two-chamber pump-action dispenser bottle a double blister (blister in blister) pack, or a wet-wipe blister containing a separate blister, containing peroxide, to be broken by force prior to application.

The kit can further comprise a container, such as a blister-pack and/or tissues, such as sanitizing wipes for applying the composition according to the present invention.

Uses

The presently disclosed composition is intended for use in cleaning, sanitizing, disinfecting and/or sterilizing a biological surface and/or a biomaterial surface in situ.

In one aspect of the invention, the antimicrobial and/or anti-viral composition according the present invention is for use in sterilizing, disinfecting and/or sanitizing a biological surface and/or a biomaterial surface in situ.

Thus, an antimicrobial and/or anti-viral composition according to the present invention is typically used for cleaning, sterilizing, disinfecting and/or sanitizing skin and/or mucosa from microbes.

In particular, the microbes to be killed or removed by the composition according to the present invention are selected from the group consisting of bacteria and virus, such as selected from the non-exclusive group consisting of Riboviria, in particular Coronaviridae (e.g., Sars-Cov-2), or Orthocoronavirinae.

An antimicrobial and/or anti-viral composition according to the present invention is in particular well-suited for killing and/or removing Coronaviruses, such as from skin and/or mucosa, including from hands, face, or oral or nasal cavities.

The present invention thus in a presently preferred aspect also relates to the application of an antimicrobial and/or anti-viral composition according to the present invention as a hand wash, oral wash, tooth wash, for nasal and/or sinus cleansing, and/or for washing virus off skin and mucosa.

The present invention thus in a presently preferred aspect also relates to the application of an antimicrobial and/or anti-viral composition according to the present invention as a hand sanitizer, mouth wash, sinus rinse, nasal rinse and/or sanitizing wipes.

The presently disclosed composition is also useful for application in peri-implant defects, and the formulation may be tailored to fit with various clinical procedures, such as but not limited to treating and preventing oral infections, for maintaining oral and/or nasal health and post-operative follow-up administrations to skin, mucosa and/or oro-laryngeal and/or nasal cavities.

The presently disclosed composition can also be used for other oral procedures, such as during surgical cleaning of periodontal defects, for preparation before regenerative procedures, in periodontal maintenance treatment, in periodontitis prophylaxis (by dental hygienists), as well as in endodontics, both in root-canal procedures and in apical surgery.

What is more, the presently disclosed composition can further be used for cleaning and/or debriding outside the oral cavity, such as, but not limited to in orthopaedic revision surgery, sanitizing of transdermal devices, in dermal wound care for cleaning of acute wounds and/or in cleaning of chronic ulcers and burns.

An antimicrobial and/or anti-viral composition according to the present invention can typically be employed for use in the treatment and/or prevention of peri-implantitis, gingivitis and/or mucositis, peri-implant mucositis and/or periodontitis or for the sanitizing of the oral cavity against contagious disease or prior to oral surgery procedures.

Periimplantitis is a typical complication related to orodental rehabilitation through the use of implants, e.g., a peri-implant disease, which is well-known to the person skilled in the art as an inflammatory reaction to oral microbes in which there is an accompanied loss of the bony support of the implant. The aetiology of the disease is conditioned by the status of the tissue surrounding the implant, implant design, degree of roughness, the poor alignment of implant components, external morphology and excessive mechanical load.

The presently described antimicrobial and/or anti-viral composition offers several approaches for an effective and rapid cleaning of an implant and/or for sanitizing or decontaminate a tissue surface in the oral cavity essentially without damaging of the delicate structure or of the implant surface and/or the tissue surface itself, and essentially without leaving contaminating material residues on the treated surface.

The invention therefore in one aspect is directed to the antimicrobial and/or anti-viral composition as defined herein and/or the kit for preparing the composition of the invention as defined herein, for use as a medical device or a medicament.

Thus, the present invention relates to the use of an antimicrobial and/or anti-viral composition according to the present invention for cleaning and/or sanitizing and/or decontaminate an implant in the oral cavity, such as an implant in situ, a tissue surface in the oral cavity, such as an outer surface of a tissue in the oral cavity, a surgically exposed surface in the oral cavity, a wound in the oral cavity, such as a wound resulting from periimplantitis or a surgical wound, a periodontal defect and/or periodontal wound, and/or an oral hard tissue defect.

Implants

The invention also relates to the use of the antimicrobial and/or anti-viral composition as defined herein and/or the kit for preparing the composition of the invention as defined herein, for the preparation of a medicament and/or a pharmaceutical and/or cosmetic composition, for cleaning and/or decontaminating an implant in the oral cavity, such as an implant in situ, a hard surface in the oral cavity, such as an outer surface of a hard tissue in the oral cavity, a surgically exposed hard surface in the oral cavity, a wound in the oral cavity, such as a wound resulting from periimplantitis or a surgical wound, a periodontal defect and/or periodontal wound, and/or an oral hard tissue defect.

The invention is also directed to the antimicrobial and/or anti-viral composition as defined herein or the kit for preparing the composition of the invention as defined herein for use cleaning, sanitizing, disinfecting, sterilizing and/or debriding an implant in the oral cavity, such as an implant in situ, a hard surface in the oral cavity, such as an outer surface of a hard tissue in the oral cavity, a surgically exposed hard surface in the oral cavity, a wound in the oral cavity, such as a wound resulting from periimplantitis or a surgical wound, a periodontal defect and/or periodontal wound, and/or an oral hard tissue defect.

Oro-Laryngeal and Nasal Medical Applications

In dentistry and otorhinolaryngology, nasal and mouth rinses are used pre- and post-surgery to lower the microbial burden during a procedure, or to treat infections. The infection of oro-larynx and nasal mucosa leads to formation of mucus that can fill up sinuses and cause nasal and sinus congestion and microbe accumulation on tongue, tonsils, teeth and gums. The invention is also directed to the antimicrobial and/or anti-viral composition as defined herein for use as mouth wash, nasal rinse and/or sinus rinse for the disinfection and cleaning of oral, laryngeal and/or nasal anatomical structures and/or surfaces.

Microorganisms

An antimicrobial and/or anti-viral composition according to the present invention is in general intended for use in cleaning, disinfecting, sanitizing and/or decontaminating a biological surface and/or a biomaterial surface in situ, e.g. for use in removal of virus, microbes, including but not limited to bacteria, yeasts, and bacterial spores, and dirt from such a biological surface and/or a biomaterial surface in situ.

Biofilms that can be prevented, eliminated and/or treated by the composition of the present disclosure include, but are not limited to, biofilms present within the oral cavity, e.g., on the surface of teeth, on the surface of mucosal/soft-tissues such as gingivae/periodontium and inside a tooth canal (e.g. the endodontic canal).

In certain embodiments, biofilms that can be prevented, eliminated and/or treated by the composition of the present disclosure include biofilms on the urinary tract, lung, gastrointestinal tract, on and/or within chronic wounds, and present on the surface (e.g., implants) and within medical devices and medical lines, e.g., catheters, medical instruments or medical tubing.

The composition of the present disclosure can be used to reduce the growth and/or inhibit the viability of one or more microorganisms, e.g., bacteria in a biofilm. For example, and not by way of limitation, the bacteria, which can be inhibited can include Streptococcus mutctns (S. mutctns), Streptococcus sobrinus, Streptococcus sctnguis (sctnguinis), Streptococcus gordonii, Streptococcus omlis, Streptococcus mitis, Actinomyces odontolyticus, Actinomyces viscosus, Aggregcttibctcter ctctinomycetemcomitctns, Ictctobctcillus spp., Porphyromoncts gingivctlis, Prevotellct intermedia, Bacteroides forsythus, Treponema denticola, Fusobacterium nucleatum, Campylobacter rectus, Eikenella corrodens, Veillonella spp., Micromonas micros, Porphyromonas cangingivalis, Haemophilus actinomycetemcomitans Actinomyces spp., Bacillus spp., Mycobacterium spp., Fusobacterium spp., Streptococcus spp., Staphylococcus aureus, Streptococcus pyogenes, Streptococcus agalectiae, Proteus mirabilis, Elebsiella pneumoniae, Acinetobacter spp., Enterococcus spp., Prevotella spp., Porphyromonas spp., Clostridium spp., Stenotrophomonas maltophilia, P. cangingivalis, Candida albicans, Escherichia coli and/or Pseudomonas aeruginosa. In certain embodiments, the bacteria are S. mutans, which is present within biofilms found in the oral cavity, e.g., on the surface of teeth.

The microorganisms most commonly associated with implant failure are spirochetes and mobile forms of Gram-negative anaerobes. Diagnosis can be based on changes of colour in the gum, bleeding and probing depth of peri-implant pockets, suppuration, x-ray and gradual loss of bone height around the tooth. The antibiotic therapy proven to be most efficacious in the antibiogram has so far been the association of amoxycillin and clavulanic acid. In addition to bacterial infections, microbial infections in the oral cavity can of course also include fungal and/or viral infections.

An antimicrobial and/or anti-viral composition according to the present invention is effective for killing bacteria, fungus and/or virus.

Typically, a composition according to the present invention is for use in cleaning skin and/or mucosa from virus such as, but not limited to, Riboviria, such as Coronaviridae, such as Orthocoronavirinae and Orthomyxoviridae such as Influenza A, B, C and D virus, Arbovirus and Isavirus, and such as Rotavirus, Norovirus, Adenovirus, Papillomavirus, Herpes virus, Hepatitis virus, Small Pox virus, Parvovirus, Ebola virus, Measles virus or Rabies virus.

In a presently preferred aspect, a composition according to the present invention is for use in cleaning skin and/or mucosa from Coronavirus or Influenza virus.

What is more, the composition described herein is antimicrobial, without causing microbial resistance, as well as anti-viral.

In consequence, the present invention relates to a method for cleaning, sanitizing, treating and/or preventing contagious diseases, infections, gingivitis and/or mucositis, peri-implant mucositis, peri-implantitis, periodontitis, tooth decay, and/or sinusitis and rhinitis, comprising cleaning of a biological surface and/or a biomaterial surface in situ by applying a composition according to the present invention to said fouled, filmed and/or contaminated structure, tissue or surface.

### EXAMPLES

The following Examples have been included to provide guidance to one of ordinary skill in the art for practicing representative embodiments of the presently disclosed subject matter.

Considering the present invention and the general level of skill in the art, those of skill can appreciate that the following Examples are intended to be exemplary only and that numerous changes, modifications, and alterations can be employed without departing from the scope of the presently disclosed subject matter.

### Example 1

Analysis of micelle formation of pluronic in presence of peroxide Pluronic® F-127 added to an aqueous solution can change its thickness depending on the concentration and temperature. F-127 is a non-ionic, surfactant polyol with a molecular weight of 12500 Daltons.

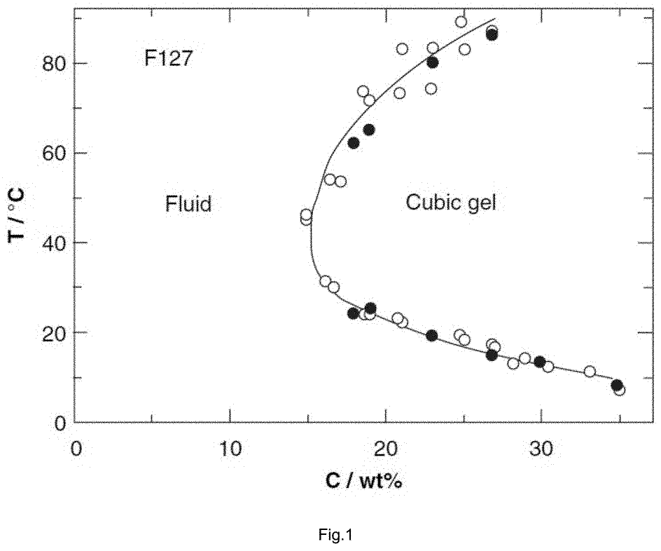

As can be seen in FIG. 1, gel boundaries for aqueous saline (physiological conditions) solutions of copolymer F-127. The filled circles are data points obtained by the tube inversion method for the mixture. The unfilled squares are data points from rheometric analyses. The chart clearly indicates that pluronic concentrations below 15% w/v in saline remains fluid independent of temperature. Fluid pluronic solutions form independent micelles, but the concentration is not high enough for the pluronic micelles to assemble into a cubic gel. Thus, low pluronic concentrations that allow for micelle formation but stay liquid and act as detergent may be good for tissue-friendly cleaning of biological surfaces, but addition of other potent reactants that can contribute to the decontamination is expected to completely disrupt the micelle formation and thus abolish the wanted detergent and denaturing effects.

Adding Peroxide to the Pluronic f-127 Gel

In order to test if micelle formation (as visualised by ability to foaming and dissolve oil) still could be obtained when mixing low concentration of Pluronic f-127 in water with various antimicrobial agents (see example 3), hydrogen peroxide, in a wide range of peroxide concentrations, was tested (Table 1).

The technique used for making aqueous poloxamer solution is simple. Weighed amount of the f-127 is slowly added to a known weight of cold water (less than 10° C.) with careful stirring. The stirring rate should be controlled so as to maintain a slight vortex in the liquid. Too rapid a stirring rate will cause aeration and the formation of foam.

TABLE 1
Compositions of suspensions containing 5% pluronic Acid F-127
(Zigma Aldrich) in water and a range of Hydrogen Peroxide (Sigma
Aldrich). After dissolving the f-127 in cold water, the right amount
of hydrogen peroxide was added, mixing was done by inverting tubes
carefully to avoid foaming, and the solutions were left on the bench
at room temperature for 10 minutes. When all the polymer has been
added, stirring can be continued (while keeping the solution cool)
until a clear solution is formed or the container can be placed in a
refrigerator and left undisturbed for several hours, at which time
solution is complete. The H₂O₂ was mixed in by slight stirring
for a few seconds when the experiment started.

| Sample | Pluronic F-127(gr) | H₂O (ml) | 50% H₂O₂ (ml) |
|---|---|---|---|
| 0 | 2.5 | 50.00 | 0.00 (0.0%) |
| 1 | 2.5 | 49.90 | 0.10 (0.1%) |
| 2 | 2.5 | 49.50 | 0.50 (0.5%) |
| 3 | 2.5 | 49.00 | 1.00 (1.0%) |
| 4 | 2.5 | 48.50 | 1.50 (1.5%) |
| 5 | 2.5 | 47.00 | 3.00 (3.0%) |
| 6 | 2.5 | 44.00 | 6.00 (6.0%) |
| 7 | 2.5 | 38.00 | 12.00 (12.0%) |

TABLE 2
After incubation all the solutions were still liquid (no gel formation
as expected) and were vortexed and the ability to form foam (as
a simple measurement of surfactant effect) was scored on a scale
from 0 (no foam) to three (much foam). As expected, sample 0 scored
maximum, and addition of hydrogen peroxide reduced foaming at
low and high concentration. Unexpectedly there were no linearity
in the reduction of foaming. In fact, samples 2 trough 4 scored
as high as the control, indicating that micelle formation and
surfactant effect were preserved in these samples. The samples
were then tested on how many oil droplets (mineral oil stained
with oil red (Sigma Aldrich)) from a needle point the solutions
could absorbed before getting turbid. Turbidity is a sign of macro-
emulsion, a stage where oil droplets become so big that they disperse
light. The positive control, Sample 0, absorbed 7 oil droplets
before getting turbid. The negative control, sample 7, turned
turbid with the first droplet. The expected linearity between
these extremes was however not observed. Surprisingly, sample
2 (0.5% Peroxide) and 3 (1.0% peroxide) actually absorbed almost
twice as many droplets than the positive control before turbidity
was observed. This indicates that Pluronic f-127 micelle formation
at low concentrations (=liquid solutions) actually is more
efficient, and dynamic, in presence of low concentrations of hydrogen
peroxide in the range of 0.5-1.5% volume by volume (in a 5% w/v
solution of f-127).

| | | Oil absorption per | |
|---|---|---|---|
| Sample | Foaming (0-3) | 50 ml (droplets) | % of H₂O₂ |
| 0 | 3 | 7 | 0.00 |
| 1 | 2 | 6 | 0.1% |
| 2 | 3 | 12 | 0.5% |
| 3 | 3 | 11 | 1.0% |
| 4 | 3 | 7 | 1.5% |
| 5 | 2 | 5 | 3.0% |
| 6 | 1 | 2 | 6.0% |
| 7 | 1 | 0 | 12.0% |

### Example 2

Test of Biocompatibility of Pluronic Acid Combined with Peroxide

Test Procedure:

The biocompatibility test was performed according to ISO 10993-5:2009 Biological evaluation of medical devices Part 5: Tests for in vitro cytotoxicity.

Colorimetric assay such MTT, WST-1 or LDH was not used as the peroxide suspension degrade the indicator colour used in these assays and thus, they cannot be used. Instead radioactive labelling with [³H] thymidine incorporation was used to analyse the cytotoxicity of the pluronic-peroxide solution on normal human dermal fibroblast cells (NHDF) (Lonza Walkersville, Inc. Walkersville, Md., USA).

Due the viscosity of the tested hydrocolloid gels (SilvaSorb® Gel, Medline Industries, Munedelein, Ill.) and the pluronic-Peroxide gels (20% Pluronic f-127 in water with 3% Hydrogen Peroxide), the test samples could not be diluted into the cell culture medium. Nor could the NHDF cells grow directly onto the gel due their lack of surface properties. To overcome this problem, the NHDF cells were grown on cell culture inserts (Millicell®, Millipore Corp., Billerica, Mass., USA) that was inserted into a well that contained the test gel diluted 1:1 against cell culture medium. The NHDF cells were cultivated in 24 well plates for 24 hours at 37 C and 5% CO2 with Dulbecco's PBS cell culture media (FGM®-2 with Insulin (CC-4021J), rhFGF-B (CC-4065J) and FBS (CC-4161J), Lonza Walkervill, Md. USA). After 24 hours growing in presence of test substances or controls (no gel and gelatin gel) 0.5 μL ³H-thymidine (PerkinElmer, Boston, USA) was added into the wells containing the cells. After 12 hours of exposure to thymidine the cells were washed three times in cold PBS and lysed with 250 μL 1M NaOH. Then 200 μL of the solubilized cell solution was transferred to 3 ml Insta-gel-2-Pluss liquid scintillation fluid. Subsequently the scintillations were counted in a liquid scintillation analyser (TRI-Corb®1500 Perkin Elmer). Results and conclusion: There were no observed difference in count number between samples exposed to pluronic-Peroxide solution and the controls indicating that the NHDF cells grew at normal rate in presence of these test gels. The hydrogen peroxide containing pluronic acid gels are safe and biocompatible. The SilvaSorb gel showed a significant decrease in count number indication slower cell growth in presence of this gel as has also been repeatedly reported in the scientific literature, indicating that this gel is slightly toxic NHDF cells.

### Example 3

Chemical Cleaning Efficacy of Liquid Pluronic f-127 Solutions Containing Peroxide

The following cleaning solutions were tested:

| Negative control | Brain Heart Infusion Broth (BHI broth) |
|---|---|
| Test 1 | H₂O₂ 3% v/v + f-127 1% w/v |
| Test 2 | H₂O₂ 5% v/v + f-127 1% w/v |
| Test 3 | H₂O₂ 3% v/v + f-127 7% w/v |
| Test 4 | H₂O₂ 5% v/v + f-127 7% w/v |
| Test 5 | Chlorhexidine 0.2% w/v + f-127 7% w/v |
| Test 6 | Ethanol 75% v/v + f-127 1% w/v |

Experimental Set-Up:

A stock Streptococcus Epidermidis culture was established in BHI broth and allowed to grow to log phase.

The S. epidermidis culture was introduced to cell culture disks of c.p. titanium and allowed to for a multilayer biofilm on the surface over a period of 2 days. After the biofilm had been established, the discs were rinsed several times in sterile, cold PBS until only adherent cells was present on the surfaces.

The discs with biofilm on were then submerged in test gel or control for 5 minutes±5 sec. making sure that the entire discs were covered by the test gels all the time. The discs were then rinsed for 10 minutes in cold PBS on a shaker, rinsed off and viewed in a table-top scanning electron microscope to see how many bacteria that were left on the test surfaces.

Results and Conclusion:

This experiment showed that cleaning efficacy increased some with increasing H₂O₂ and increasing Pluronic f-127 concentration. The difference between test 1 and test 4 was however surprisingly small and not statistically significant, as all four tests performed very well and almost no bacteria remained on the surface. The negative control was as expected completely covered by bacteria with no effect of the PBS washing. The positive Chlorhexidine and Ethanol controls was surprisingly ineffective. Almost all of the biofilm was retained on the surfaces in these two groups. Later analysis with regrowth of bacteria from these surfaces revealed that all bacteria in these two tests were dead, but that the bulk of the biofilm remained attached to the surface, forming a contaminated layer that provide a strong bridgehead for new biofilm formation if a new contamination occurs.

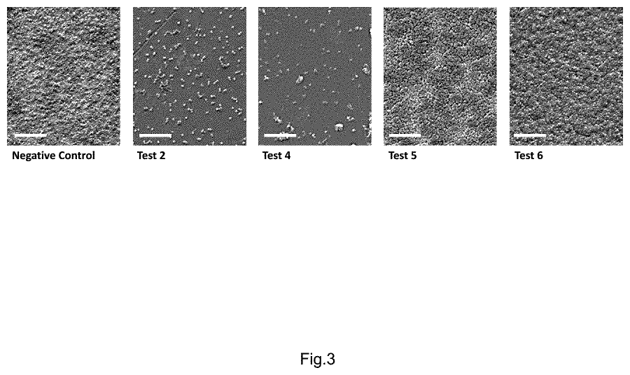

The surprising finding in this study is that it seems to be a synergistic effect between hydrogen peroxide and Pluronic f-127 that not only kills bacteria but also disintegrate and solubilize the entire biofilm, leaving an almost totally decontaminated surface (FIG. 3). The concentration of the peroxide and pluronic can be reduced toward 3% hydrogen peroxide in 1% pluronic acid without losing much effect compared to stronger solutions. This also indicates that the synergy between hydrogen peroxide and pluronic acid can be fine-tuned and probably has an optimal concentration-relation that allows for rapid and dynamic micelle (detergent and denaturation) effect and at the same time maintains the effectiveness of activated oxygen release that kills microbes and dissolves organic molecules. This optimal formulation appears to be lower in concentration of both peroxide and pluronic when the components are mixed together than what one would expect based on the effect of the separate components.

As is shown in FIG. 3, titanium surfaces contaminated with S. epidermidis bacteria was cleaned with growth medium (BHI, Negative control), H₂O₂ 5% v/v+f-127 1% w/v (Test 2), H₂O₂ 5% v/v+f-127 7% w/v (Test 4), Chlorhexidine 0.2% w/v+f-127 7% w/v (Test 5) or Ethanol 75% v/v+f-127 1% w/v. The negative control did not remove the bacteria, nor was the bacteria destroyed. The peroxide+pluronic acid mix killed all bacteria, dissolved the biofilm and removed the debris almost completely (Tests 2 & 4). Test 4 was slightly, but not statistically significant, more effective than Test 2. Chlorhexidine (Test 5) and Ethanol (Test 6) did kill most of the bacteria but did not remove any debris from the surface, leaving the surfaces heavily contaminated. Surfaces visualized after cleaning in a scanning electron microscope. Scale bar is 20 micrometres.

### Example 4

Staining of Living and Dead Bacteria on Decontaminated Surfaces

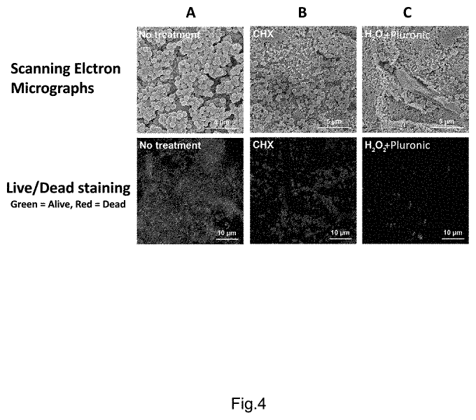

Titanium surfaces contaminated with S. epidermidis bacteria and grown to biofilm in BHI broth was removed from growth medium and submerged in sanitizing solution for 5 minutes on a slow shaker. Discs were sanitized with A) saline (Negative control), B) Chlorhexidine 0.2% w/v, or C) H₂O₂ 3% v/v+f-127 pluronic Acid 5% w/v. After cleaning the surfaces were stained with fluorescent dyes, green for living microbes and red for dead microbes. The negative control (saline) did not remove any of the microbes, nor was the biofilm removed. The chlorhexidine killed all microbes but left the dead cells to contaminate the surface. The hydrogen peroxide+pluronic acid mix killed all microbes, dissolved the biofilm and removed the contamination almost completely. Surfaces were visualized in a confocal microscope after cleaning and staining (FIG. 4).

### Example 5

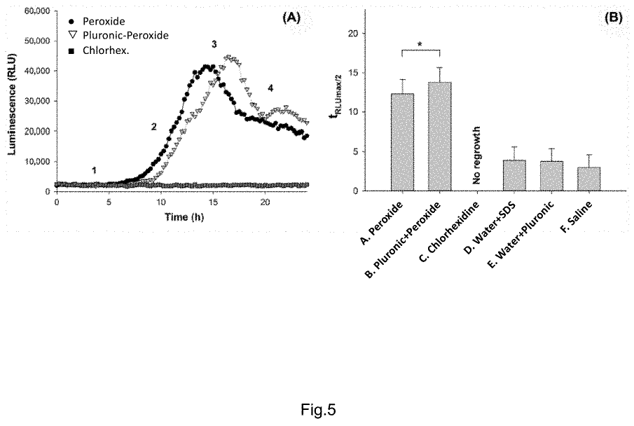

Regrowth of S. epidermidis after sanitizing a contaminated titanium surface Regrowth of S. epidermidis bacteria harbouring the luciferase gene making them fluorescent, on sanitized titanium surfaces. Surfaces was first contaminated with bacteria and incubated in BHI broth until the surfaces were completely covered in biofilm. The titanium discs were then submerged in sanitizing solution and left on a slow shaker for 5 minutes. After sanitizing the discs were rinsed in a large volume of sterile water, then covered by BHI medium again and incubated at 37 centigrade. At hourly intervals the discs were placed in a fluorometer to measure regrowth of bacteria by amount of fluorescent as compared to an untreated calibrated control and a sterile disc. The results show (see FIG. 5B) that D) water with detergent (SDS 0.1% w/v), E) water with 5% pluronic acid and F) saline had minimal sanitizing effect, as regrowth of the biofilm occurred within 3-4 hours after cleaning. Chlorhexidine, 0.2%, on the other hand killed all bacteria with no regrowth observed although the dead biofilm stuck and remained on the surface (see FIGS. 3 and 4). Hydrogen peroxide, 5% v/v, alone (A) cleaned the surface well as seen by a delay of about 13 hours before the biofilm restored itself. However, hydrogen peroxide 3% v/v combined with 5% pluronic acid in water cleaned the surface significantly better, delaying regrowth with more than 15 hours (see also FIG. 5A).

### Example 6

Pluronic with Peroxide is More Skin-Friendly than an Alcohol Containing Hand-Sanitizer

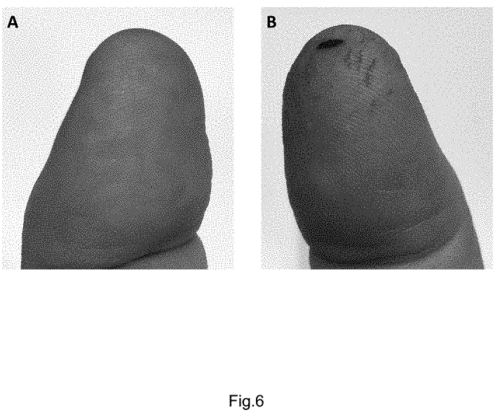

Left and right thumbs were washed and sanitized four times daily for five consecutive days with a mix of A) pluronic acid (5% w/v) and hydrogen peroxide (3% v/v) or B) Antibac Pharma™ containing ethanol and propan-2-ol respectively. After completion of the 5th day sanitizing procedure both fingers were stained with a solution of Indian Red (iron oxide) to visualize fissures and cracks in the skin. After staining the fingers were washed thoroughly in warm water with soap, left to air dry and photographed in daylight. Fissures and cracks in the skin of the thumbs are easily visible as striated brown staining. The Antibac Pharma™ treated finger show deep fissures and cracks with callous, dry and hard skin. The pluronic-peroxide formulation however, caused no harm to the skin of the left thumb, leaving skin intact and tender. This demonstrate that the pluronic-peroxide formulation is more biocompatible and tissue friendly than alcohol-based hand sanitizers (FIG. 6).

### Example 7

Efficiency of Sanitizing Solutions on Removing Organic Contaminants

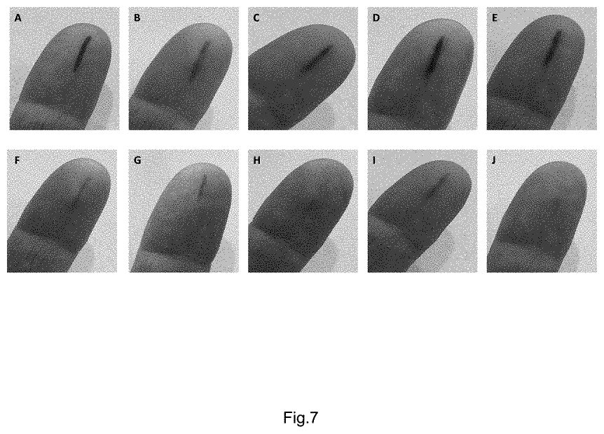

To demonstrate the ability of hand sanitizers and cleaning solutions to remove stains and contamination from skin, a panel of products and solutions were tested against stains of permanent ink. Fingers were marked with a line of permanent blue ink (Lyreco™ permanent marker for laboratory use). The ink was applied with a pen and left to air-dry for approx. 5 minutes. The fingers were then cleaned with the various sanitizing solutions for about 6-8 seconds, mimicking the normal application time for hand sanitizers in daily life. The sanitizing agent was thoroughly wiped off with paper tissue and the fingers were photographed with an iPhone X in daylight using the automatic exposure control. The intensity of the remaining stain is regarded to be an inverse visual gauge for the cleaning efficacy of the tested solutions. Finger A) was washed only with water. Finger B) was cleaned with warm water and soap. Finger C) was cleaned with 0.2% chlorhexidine and finger D) was rinsed with Pyrispet™ (0.1% cetyl-pyridinium chloride). Finger E) was cleaned with Antibac Pharma™ (Alcohol based sanitizer containing ethanol and propan-2-ol), finger F) was cleaned with bleach (4% Sodium hypochlorite). Finger G) and H) was rinsed with hydrogen peroxide (1% and 5% v/v in water respectively). Finger 1) was washed in a solution of pluronic acid f-127 in water (5% w/v). Finger J) was sanitized with a mix of pluronic acid (5% w/v) and hydrogen peroxide (1% v/v). The results show that bleach and hydrogen peroxide is very effective in removing the permanent ink stain. However, surprisingly, the most efficient removal of the stain is obtained by the combination of pluronic acid and weak hydrogen peroxide (FIG. 7).

### Example 8

Application for Skin Sanitizing, Two-Chamber Dispenser Bottle.

The two-component pluronic-Peroxide solution described here is intended for hand sanitizing after possible exposure to virus contamination. Virus are genetic code (RNA or DNA) wrapped in a protein envelope to protect and transmit the genetic information. Most sanitizers only attack (denature or fix) the protein envelope so that the virus is no longer very contagious, but do not remove the virus per se or attack the virus genome. The combination of peroxide and pluronic do both. The peroxide degrades both proteins and RNA and DNA, and the pluronic also denatures proteins and dissolves the organic material into its micelle structure, completely destroying and removing the viral particles from the skin, together with other contaminants and dirt.

A 500 ml dispenser bottle is divided in two chambers; one containing 400 milliliters of f-127 pluronic acid at 10% w/v in water, and one chamber containing 100 milliliters of Hydrogen Peroxide 25% v/v in water. A pump with two inlets pumps liquid from both chambers into the dispenser tip for mixing and application. The mixed solution is evenly distributed over the hands as with any other sanitizer, left to work for some seconds and is then rinsed off under running tap water. There is no need to apply any soap as the liquid itself also work as a detergent. The clean hands are dried off with a paper towel or air dried. No extraordinary precautions or actions are necessary to remove the solution. The solution is completely harmless after rinsing and will decompose to water, oxygen and carbon dioxide with no environmental footprint or impact.

### Example 9

Application for Skin Sanitizing, Dispenser Bottle with Emulsion.

The two-component pluronic-peroxide solution described here is intended for hand sanitizing after possible exposure to virus contamination. Virus are genetic code (RNA or DNA) wrapped in a protein envelope to protect and transmit the genetic information. Most sanitizers only attack (denature or fix) the protein envelope so that the virus is no longer very contagious, but do not remove the virus per se or attack the virus genome. The combination of peroxide and pluronic do both. The peroxide degrades both proteins and RNA and DNA, and the pluronic also denatures proteins and dissolves the organic material into its micelle structure, completely destroying and removing the viral particles from the skin, together with other contaminants and dirt.

A 500 ml dispenser bottle contains a biphasic emulsion in two equal parts. One part of the emulsion contains f-127 pluronic acid in an oily suspension (10% w/v). Etheric oils or perfume can be added for pleasant smell. The other phase contains a water solution with hydrogen peroxide (10% v/v). Before application, the bottle is shaken vigorously and a pump with dispenser tip is used to apply the emulsion mix onto hands. The emulsion is evenly distributed over the hands as with any other sanitizer, left to work for some seconds and is then rinsed off under running tap water or simply left to air dry. There is no need to apply any soap as the liquid itself also work as a detergent. No extraordinary precautions or actions is necessary to remove the solution. The solution completely harmless after rinsing and will decompose to water, oxygen and carbon dioxide with no environmental footprint. The remaining oils will protect the skin from drying and make it smooth.

### Example 10

Application for Skin Sanitizing, Blister-In Blister Pack

The two-component pluronic-peroxide solution described here is intended for hand sanitizing after possible exposure to virus contamination. Virus are genetic code (RNA or DNA) wrapped in a protein envelope to protect and transmit the genetic information. Most sanitizers only attack (denature or fix) the protein envelope so that the virus is no longer very contagious, but do not remove the virus per se or attack the virus genome. The combination of peroxide and pluronic do both. The peroxide degrades both proteins and RNA and DNA, and the pluronic also denatures proteins and dissolves the organic material into its micelle structure, completely destroying and removing the viral particles from the skin, together with other contaminants and dirt.

A 1 ml blister pack is contained inside another 5 ml blister pack. The inner blister pack contains hydrogen peroxide (25% v/v), the outer blister pack contains 3 ml f-127 pluronic acid in perfumed water (10% w/v) and the first blister pack. When needed the inner blister pack is ruptured by applying manual force, then mixed by squeezing the outer pack. After mixing the outer blister pack is opened and the content is evenly distributed over the hands as with any other sanitizer, left to work for some seconds and is then rinsed off under running tap water. There is no need to apply any soap as the liquid itself also work as a detergent. The clean hands are dried off with a paper towel or air dried. No extraordinary precautions or actions is necessary to remove the solution. The solution completely harmless after rinsing and will decompose to water, oxygen and carbon dioxide with no environmental footprint or impact.

### Example 11

Application for Skin Sanitizing, Wet Wipes

The two-component pluronic-Peroxide solution described here is intended for hand sanitizing after possible exposure to virus contamination. Virus are genetic code (RNA or DNA) wrapped in a protein envelope to protect and transmit the genetic information. Most sanitizers only attack (denature or fix) the protein envelope so that the virus is no longer very contagious, but do not remove the virus per se or attack the virus genome. The combination of peroxide and pluronic do both. The peroxide degrades both proteins and RNA and DNA, and the pluronic also denatures proteins and dissolves the organic material into its micelle structure, completely destroying and removing the viral particles from the skin, together with other contaminants and dirt.

A 1 ml blister pack is contained inside another 5 ml blister pack. The inner blister pack contains hydrogen peroxide (25% v/v), the outer blister pack contains 3 ml f-127 pluronic acid (10% w/v) in perfumed water, a folded paper tissue and the first blister pack. When needed the inner blister pack is ruptured by applying manual force, then mixed by squeezing the pack. After mixing, the outer blister pack is opened and the folded paper tissue, now activated with the peroxide-pluronic mix used to wipe the skin, hands or contaminated surfaces free of dirt, microbes and viral contaminants. There is no need to apply any soap as the liquid itself also work as a detergent. The clean hands, skin or surface is left to dry in air.

No extraordinary precautions or actions is necessary to remove the solution. The solution completely harmless after rinsing and will decompose to water, oxygen and carbon dioxide with no environmental footprint or harm to the skin.

### Example 12

Mouth Rinse Application for Oral Sanitizing, Dispenser Bottle with Emulsion

The two-component pluronic-peroxide solution described here is intended for oral sanitizing for microbial control or during a contagious viral pandemic. Virus are genetic code (RNA or DNA) wrapped in a protein envelope to protect and transmit the genetic information. Most viral sanitizers only attack (denature or fix) the protein envelope so that the virus is no longer very contagious, but do not remove the virus per se or attack the virus genome. The combination of peroxide and pluronic does both. The peroxide degrades both proteins and RNA and DNA, and the pluronic also denatures proteins and dissolves the organic material into its micelle structure, completely destroying and removing the viral particles from the oral cavity and throat, together with other microbes, contaminants and mucus.

A 1000 ml dispenser bottle is divided in two chambers; one containing 800 milliliters of f-127 pluronic acid at 5% w/v in saline with sodium fluoride and taste and smell modifiers, and one chamber containing 200 milliliters of hydrogen peroxide 16% v/v in water. A pump with two inlets is used to pumps liquid from both chambers into the dispenser tip for mixing and application. 10 milliliters of the mixed solution are used to rinse the mouth and throat for about 1 minute, and then spat out in the sink. There is no need to brush teeth or rinse with water after application as the liquid itself is harmless to humans, but it is no harm in doing it either. No extraordinary precautions or actions is necessary to remove the solution. The solution completely harmless after rinsing and will decompose to water, oxygen and carbon dioxide with no environmental footprint or health impact.

The mouthwash can also be used against oral infections periodontitis and peri-implantitis and tooth decay, both prophylactically and for post-surgery infection control and maintenance treatment after periodontal or implant-related procedures. It can also be used to clean toothbrushes and intra-oral devices (dentures, anti-snoring devices, orthodontic appliances etc.) to avoid re-infection from repeated use.

### Example 13

Nasal/Sinus Rinse

Nasal and sinus rinse applications, disposable bottles with applicator The two-component pluronic-peroxide solution described here is intended for nasal/sinus rinsing against a rhinoviral infection causing congestion or for virus removal during a contagious flu or corona virus pandemic. Virus are genetic code (RNA or DNA) wrapped in a protein envelope to protect and transmit the genetic information. Most viral sanitizers only attack (denature or fix) the protein envelope so that the virus is no longer very contagious, but do not remove the virus per se or attack the virus genome. The combination of peroxide and pluronic does both. The peroxide degrades both proteins and RNA and DNA, and the pluronic also denatures proteins and dissolves the organic material into its micelle structure, completely destroying and removing the viral particles from the nasal cavity and sinuses, together with other contaminants and mucus.

Fifty milliliters of hydrogen peroxide (5% v/v in saline) is added to a 250 ml disposable, soft (squeezable) bottle with nasal applicator top containing 200 milliliters of f-127 pluronic acid at 1.25% w/v in saline and mixed by vigorous shaking. The head is placed over a sink, and the bottle containing the mix is then placed against one nostril and squeezed carefully until the fluid comes out of the other nostril and goes into the sink below. The procedure is repeated until the bottle is empty. Blow the nose after application is completed. There is no need to rinse with after application, the liquid itself is harmless to humans. No extraordinary precautions or actions is necessary to remove the solution. The solution completely harmless after rinsing and will decompose to water, oxygen and carbon dioxide with no environmental footprint or health impact.

The nasal/sinus rinse can also be used against nasal infections or against acute and chronic sinusitis. The solution can also be used both prophylactically and for post-surgery infection control after otorhinolaryngeal procedures.

1. A composition, comprising an antimicrobial and/or anti viral mixture consisting of:
(a) H₂O₂ at a final concentration of between 3.0-7.0% v/v, and
(b) a poloxamer at a concentration of 0.1-10.0% w/v.

2. The composition according to claim 1, wherein the poloxamer is at a concentration of 1.0-5.0% w/v.

3. The composition according to claim 2, wherein the poloxamer is at a concentration of at the most 5.0% w/v.

4. The composition according to claim 1, wherein the component (b) is a composite hydrogel formulation.

5. The composition according to claim 1, wherein the poloxamer is a mixture of poloxamers.

6. The composition according to claim 1, wherein the component (b) is selected from the group consisting of pluronic acid, Pluronic® F-127 and Poloxamer 407.

7. The composition according to claim 1, wherein the H₂O₂ of component (a) has a final concentration of 3.0% v/v.

8. The composition according to claim 1, wherein component (a) and component (b) are provided in a 1:1 ratio.

9. The composition according to claim 1, further comprising water and/or physiological saline.

10. The composition according to claim 1, wherein components:
(a) H₂O₂, and
(b) a poloxamer,
are kept separate from each other until use.

11. The composition according to claim 1, further comprising an additional bioactive substance.

12. The composition according to claim 11, wherein the bioactive substance is selected from the group consisting of EMD, peptides, drugs, bioactive ions like fluoride, small molecules, radioactive molecules, antimicrobial molecules and radio-opaque molecules or any combination thereof.

13. The composition according to claim 1, further comprising an additional antimicrobial substance.

14. The composition according to claim 1, wherein the composition has a shelf-life of at least 1 years at room temperature.

15. A method of sanitizing, sterilizing, disinfecting and/or decontaminating a biological surface and/or a biomaterial surface comprising applying the composition according to claim 1 to a biological surface and/or a biomaterial surface.

16. A method according to claim 15, wherein the biological surface is skin and/or mucosa of a subject.

17. The method according to claim 15, wherein the microbes comprise bacteria or virus or both.

18. The method according to claim 17, wherein the microbes comprise Riboviria.

19. The method according to claim 17, wherein the microbes comprise Coronaviridae, Orthomyxoviridae, Caliciviridae or Reoviridae or any combination thereof.

20. The method according to claim 17, wherein the microbes comprise Orthocoronavirinae.

21. The method according to claim 17, wherein the microbes comprise Coronavirus, Rotavirus, Norovirus or Influenza virus type A, B, C or D or any combination thereof.

22. The method according to claim 17, wherein the microbes comprise Sars-Cov-2.

23. A hand wash, oral wash, nasal cleansing solution, sinus cleansing solution, or skin cleansing solution comprising the composition of claim 1.
